(19) United States
(12) Patent Application Publication
(10) Pub. No.: US 2010/0305509 A1
(43) Pub. Date: Dec. 2, 2010

(54) VASCULAR INTRODUCER SHEATH WITH MULTIPLE PEEL LINES

(75) Inventors: Thomas P. Osypka, Palm Harbor, FL (US); Timothy L. Sass, SR., Port Richey, FL (US); Jeff Jeffrey Drum, Palm Harbor, FL (US)

Correspondence Address: EDWARDS ANGELL PALMER & DODGE LLP, P.O. BOX 55874, BOSTON, MA, 02205, US

(73) Assignee: Oscor Inc., Palm Harbor, FL (US)

(21) Appl. No.: 12/790,034

(22) Filed: May 28, 2010

Related U.S. Application Data
(60) Provisional application No. 61/182,662, filed on May 29, 2009.

(51) Int. Cl. A61B 17/34

(52) U.S. Cl. 604/164.05

(57) ABSTRACT

A peelable vascular introducer including an elongated tubular sheath having opposed proximal and distal ends and at least three circumferentially spaced peel lines extending from the proximal end to the distal end. The peelable vascular introducer also includes spreadable handles associated with the proximal end of the sheath configured to split the sheath along the peel lines.

### CROSS-REFERENCE TO RELATED APPLICATIONS

This application claims priority to U.S. Provisional Patent Application No. 61/182,662, filed May 29, 2009, which is incorporated herein by reference in its entirety.

### BACKGROUND

The present invention relates to vascular introducers. Particularly, the present invention relates to peelable vascular introducers, removable by peeling such as from a guide wire following insertion thereof into a blood vessel (e.g. a vein) of a patient.

Introducer devices have been employed for inserting catheters, guide wires, leads and the like into patients. A typical procedure provides for insertion of a dilator or needle encased within a sheath into the vasculature of a patient. After insertion, the dilator or needle may be removed leaving the sheath protruding from the patient's vein. The sheath is then removed. Typically, to remove the sheath, it may be split or peeled away along two diametrically opposed score lines. Alternatively, the sheath may be slit open using a specialized cutting blade.

Examples of splittable or peelable introducer sheaths are shown in U.S. Pat. No. 6,494,860 to Rocamora et al., U.S. Pat. No. 7,192,433 to Osypka et al., U.S. Pat. No. 6,892,087 to Osypka, U.S. Patent Application Publication No. 2009/0030374 to Osypka, and U.S. Design Pat. No. D576,276 to Osypka. Each of these documents is incorporated herein by reference in its entirety.

Typically, splittable introducer sheaths are prepared by forming straight score lines along the length of the sheath using a mandrel. An example of a device for slitting an introducer sheath is disclosed in U.S. Pat. No. 4,687,469 to Osypka, the disclosure of which is also incorporated herein by reference in its entirety. Typical slitter devices include a body or handle portion with a cutting edge or knife secured thereto such that the cutting edge may be manually guided to cut the sheath.

Drawbacks to typical peelable sheaths include irregular splitting of the sheaths if two halves are not pulled apart relatively symmetrically. In such situations, the sheath can be torn and not peel cleanly along the preformed lines. In this case, the sheath must typically be removed by cutting.

Further, the separation line of splittable handles (as shown, for example, in U.S. Pat. No. D576,276) may be imperfectly aligned with the peel lines of conventional sheaths. If alignment is not sufficiently precise, when the user breaks the hub of the introducer handle, the sheath will not split. In this case, the sheath also must be removed by cutting. When forced to cut sheaths in the event of uneven separation, the expense of a separate device can be significant. Additionally, the manual dexterity required to operate a slitter can be undesirably high. There is a need, therefore, for an improved sheath which eliminates the drawbacks of the aforementioned introducer sheaths.

### SUMMARY OF THE INVENTION

The purpose and advantages of the present invention will be set forth in and apparent from the description that follows. Additional advantages of the invention will be realized and attained by the methods and systems particularly pointed out in the written description and claims, as well as from the appended drawings.

To achieve these and other advantages and in accordance with the purpose of the invention, as embodied, the invention includes a peelable vascular introducer including an elongated tubular sheath having opposed proximal and distal ends and at least three circumferentially spaced peel lines extending from the proximal end to the distal end. The peelable vascular introducer also includes spreadable handles associated with the proximal end of the sheath configured to split the sheath along the peel lines.

In accordance with another embodiment of the invention, a peelable vascular introducer includes an elongated tubular sheath having opposed proximal and distal ends, and at least three circumferentially spaced peel lines extending from the proximal end portion to the distal end portion with an axially extending pitch. The peelable vascular introducer also includes spreadable handles associated with the proximal end of the sheath for facilitating splitting of the sheath along the peel lines.

The peelable vascular introducer can include any efficacious number of peel lines. For example, in accordance with an exemplary embodiment, between 8 and 20 peel lines are provided. In an exemplary embodiment, between 10 and 14 peel lines are provided. In one exemplary embodiment, 12 peel lines are provided. In accordance with an exemplary embodiment, a sheath can have a diameter of about 4 French to about 22 French. In accordance with one embodiment, 12 peel lines are provided in a sheath having a diameter of about 7 French. Accordingly, it may be desirable to provide larger numbers of peel lines in larger introducer sheaths. The peel lines are, in accordance with one exemplary embodiment, spaced regularly about the circumference of the sheath. The peel lines can be defined by areas of reduced material thickness formed in the sheath, circumferentially arranged between areas of greater thickness. Alternatively or additionally, peel lines can be defined in the sheath by scored regions and/or by perforated regions.

The sheath can be formed from any suitable material, including but not limited to polyether block amide material.

The peelable vascular introducer can also include peel lines with an axially extending pitch of between five degrees and forty-five degrees.

It is to be understood that both the foregoing general description and the following detailed description are for the purpose of providing understanding of the invention by providing examples of preferred embodiments.

### BRIEF DESCRIPTION OF THE DRAWINGS

The accompanying drawings, which are incorporated in and constitute part of this specification, are included to illustrate and provide a further understanding of the devices of the invention. Together with the description, the drawings serve to explain the principles of the invention, wherein:

FIG. 1 is a perspective view of a percutaneous vascular introducer sheath with a handle assembly constructed in accordance with an exemplary embodiment of the invention;

FIG. 2 is a perspective view of the sheath and handle assembly of FIG. 1, illustrating the plurality of peel lines of the introducer sheath and the handle assembly;

FIG. 3 is a cross-sectional view of the sheath taken along the line 3-3 of FIG. 2, illustrating the plurality of peel lines of the introducer sheath;

FIG. 3A is a detailed perspective view of the sheath taken from a portion of the area 3A shown in FIG. 3, illustrating the plurality of peel lines of the introducer sheath;

FIG. 4 is a perspective view of the sheath and handle assembly of FIG. 1 constructed in accordance with an embodiment of the invention, illustrating the plurality of peel lines of the introducer sheath with an axially extending pitch and the handle assembly;

FIG. 5 is a cross-sectional view of an introducer sheath having five peel lines;

FIG. 6 is a cross-sectional view of an introducer sheath having sixteen peel lines;

FIG. 7 is a cross-section view of an introducer sheath having eight peel lines and a plurality of indentations on the outer surface of the sheath;

FIG. 8 is a perspective view of the vascular introducer sheath of FIG. 1 being percutaneously introduced into a blood vessel along a guidewire;

FIG. 9 is a perspective view of a portion of the sheath and handle assembly of FIG. 1, illustrating the sheath being separated by spreading the handle assembly along a pair of peel lines;

FIG. 9A is cross-sectional view of the sheath taken along the line 9a-9a of FIG. 8, illustrating the sheath being separated by a pair of peel lines perpendicular to line 9a-9a;

FIG. 10 is a perspective view of the sheath of FIG. 1, illustrating the sheath being separated along a pair of peel lines with a tear line crossing over to an alternate peel line;

FIG. 10A is a cross-sectional view of the sheath taken along the line 10a-10a of FIG. 10, illustrating the peel line separation after a tear line between peel lines; and

FIGS. 11A-11F illustrate various exemplary peel line configurations and geometries in accordance with the invention.

### DETAILED DESCRIPTION

Reference will now be made in detail to the presently preferred embodiments of the invention, examples of which are illustrated in the accompanying drawings.

The subject introducer sheaths overcome many of the prior art problems associated with removing introducer sheaths. The advantages, and other features of the technology disclosed herein, will become more readily apparent to those having ordinary skill in the art.

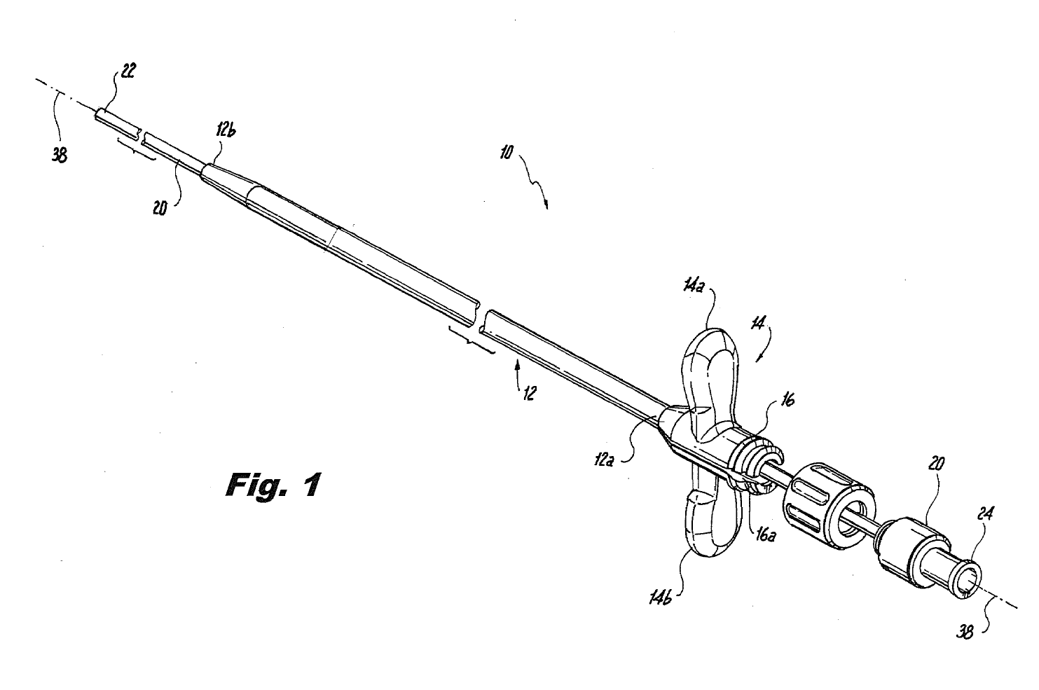

Referring now to FIG. 1, there is illustrated a vascular introducer assembly 10 that includes an elongated tubular sheath 12 defining opposed proximal and distal end portions 12a, 12b. A handle assembly 14 is operatively associated with the proximal end portion 12a of the tubular sheath 12. The handle assembly 14 includes a central hub 16 with a central parting line 16a defining a pair of opposed radially outwardly extending spreadable/separable handles 14a, 14b.

The introducer assembly 10 further includes an elongated dilator 20 having a distal end portion 22 and a proximal luer lock fitting 24. Although not shown explicitly, the central hub 16 of the handle assembly 14 can be adapted and configured to threadedly receive the dilator 20, as disclosed in U.S. Pat. No. 7,192,433, which is incorporated herein by reference in its entirety.

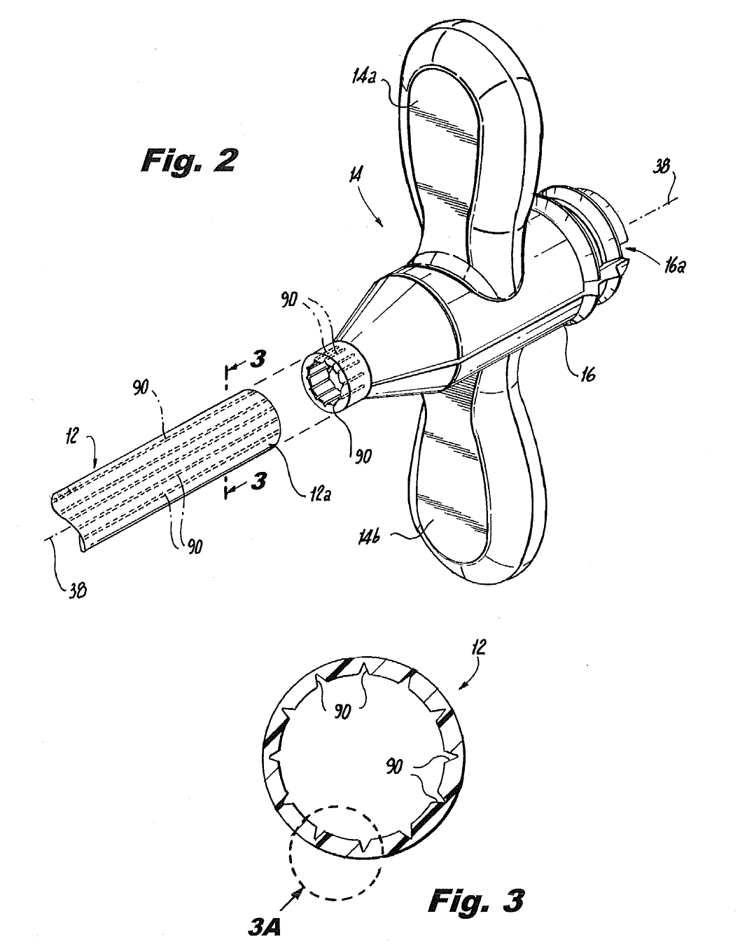

Referring to FIG. 2, there is illustrated a perspective view of the sheath and handle assembly 14 of FIG. 1, illustrating the plurality of peel lines 90 of the introducer sheath 12. In one embodiment, the plurality of peel lines 90 are disposed substantially within the inner perimeter of the sheath 12 generally parallel to a longitudinal axis 38 of the sheath.

Referring to FIG. 3, there is illustrated a cross-sectional view of the peelable introducer sheath taken along line 3-3 of FIG. 2. The tubular sheath 12 includes a plurality of peel lines 90, that extend generally parallel to a longitudinal axis 38 of the sheath 12 from the proximal end 12a to the distal end 12b as shown in FIG. 1. The peel lines are relatively numerous as compared with typical vascular introducers, which have only two. In the exemplary embodiment illustrated in FIG. 3, sheath 12 includes twelve peel lines 90 are provided. In one exemplary embodiment, the peel lines 90 can be generally parallel to the longitudinal axis 38 as shown in FIG. 2, the peel lines 90 can alternatively be provided with an axially extending pitch, at least partially wrapping about the longitudinal axis 38 of the sheath 12, if desired. If so embodied, the axially extending pitch of the peel lines 90 is preferably between about 5 degrees and about 45 degrees, although variations well outside this range are also contemplated.

FIG. 3A illustrates a detailed perspective view of the sheath taken from reference area 3A shown in FIG. 3. In one exemplary embodiment, the depth of the peel lines 90 are approximately half of the total thickness of the sheath 12 material.

FIG. 4 is a perspective view of the sheath and handle assembly of FIG. 1 constructed in accordance with an embodiment of the invention, illustrating the plurality of peel lines of the introducer sheath with an axially extending pitch and the handle assembly. In one embodiment, the plurality of peel lines 90 are disposed substantially within the inner perimeter of the sheath 12 and are axially pitched along the longitudinal axis 38 of the sheath. The axially extending pitch of the peel lines 90 can be between about 5 degrees and 45 degrees in reference to the longitudinal axis 38 of the sheath.

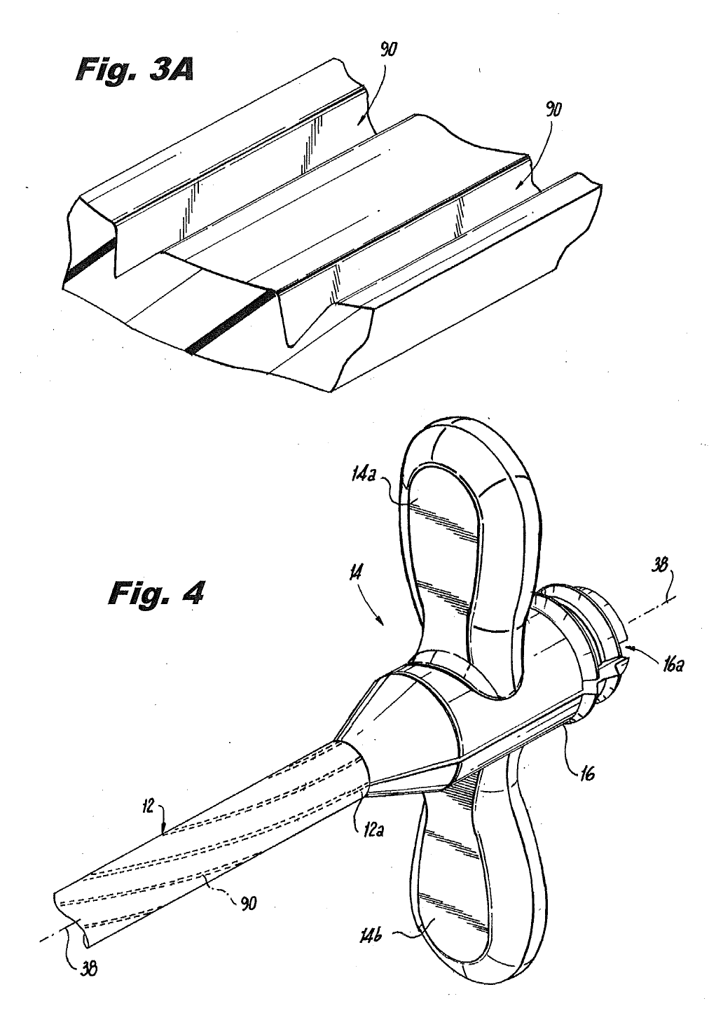

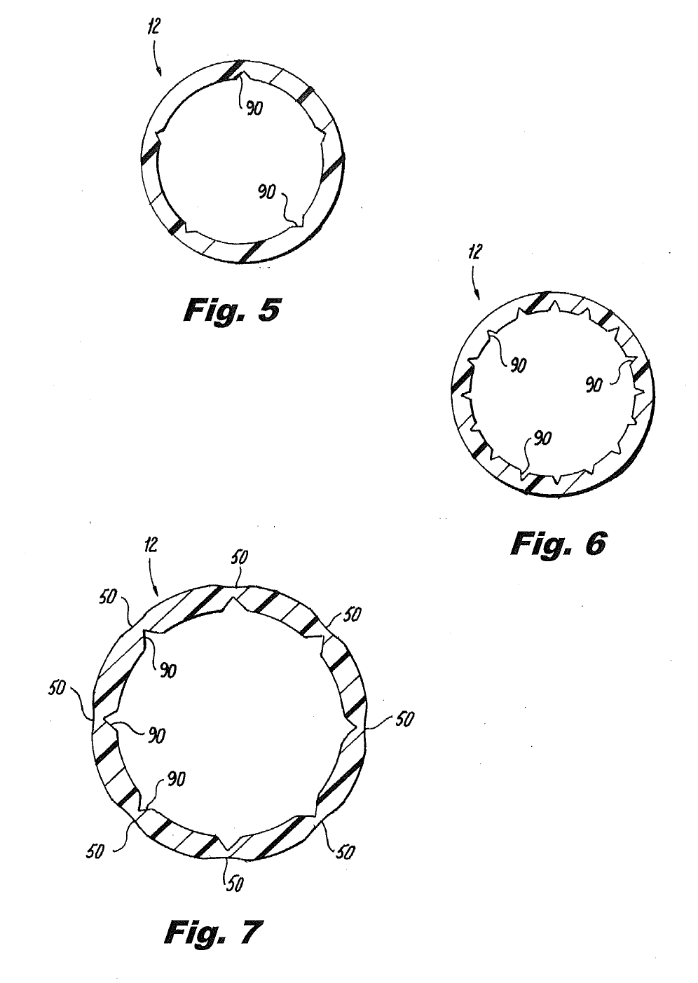

FIG. 5 illustrates an exemplary embodiment of sheath 12 having five peel lines 90. FIG. 6 illustrates an exemplary embodiment of sheath 12 having sixteen peel lines 90. FIG. 7 illustrates an exemplary embodiment of sheath 12 having eight peel lines 90. In this embodiment, a further area of weakness at the peel lines 90 is shown with indentions 50 having a generally rounded shape located on the outer surface of sheath 12. It is to be understood, however, that any number of peel lines 90 can be provided in accordance with the invention, including but not limited to any number between three and twenty-two. In one exemplary embodiment, twelve peel lines are provided in a sheath having a diameter of about 7 French.

The peel lines are lines of weakness that can be formed by forming the sheath 12 with thinner material regions corresponding to the peel lines, or by scoring or perforating the sheath in those areas during or after manufacture. As compared to prior devices having two peel lines, if the sheath 12 begins to tear outside of the peel line 90, the tearing can continue a short distance to the next peel line where it will again follow a predetermined route to allow removal of the sheath 12. Accordingly, removal of the sheath by cutting is not necessary.

When sheath 12 constructed in accordance with the invention is over-molded with break-away handles, as with handles 14a, 14b of FIG. 1, it is not critical that the parting line 16a of the handle assembly 14 be precisely aligned with any of the peel lines 90. When peeling the sheath 12, removal can be completed regardless of the rotational orientation of the handle parting line 16a with respect to the peel lines 90. If the actual peel line runs outside of the designated weakened peel line area, the actual peel line tear will continue until it runs into the next peel line 90.

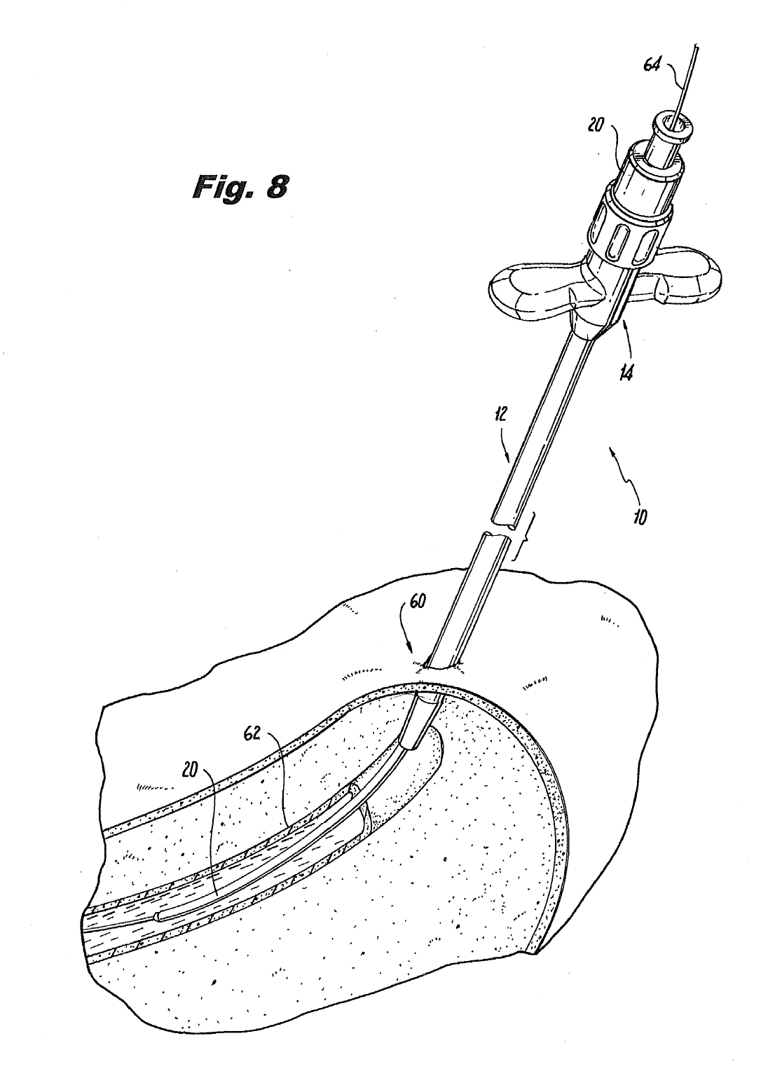

FIG. 8 shows a perspective view of the introducer sheath 12 of FIG. 1, illustrating the sheath 12 after insertion through a patient's skin 60 and into the patient's blood vessel 62. Insertion of the vascular introducer 10 is typically accomplished by first inserting a needle (not shown) into the blood vessel 62 at a desired location. The position of the needle is verified by observing fluid return or by a similar method. While the needle is held firmly in place, a guidewire 64 is inserted through the needle caimula (not shown) to the desired depth. The guidewire 64 is then held in place and the needle is withdrawn. Pressure is then applied on the puncture site in order to minimize blood loss. Next, the introducer assembly 10 is threaded over the guide wire 64. The introducer assembly 10 is grasped close to the skin surface 60 and advanced to the desired position within the blood vessel 62. Then, the dilator 20 and the guidewire 64 are removed, leaving the sheath 12 installed. A lead, catheter, or similar diagnostic or therapeutic device is then introduced into the sheath and advanced to the desired position within the blood vessel 62. Lastly, the sheath 12 is removed, leaving the device disposed within the blood vessel of the patient. As described below, the sheath 12 may be split or peeled away.

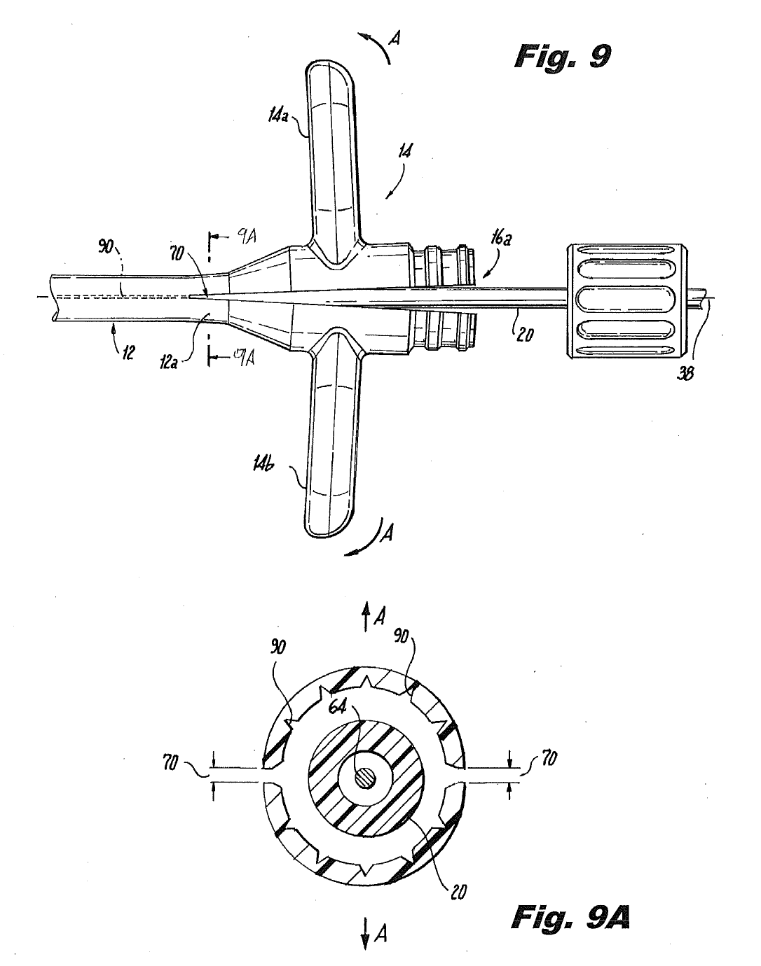

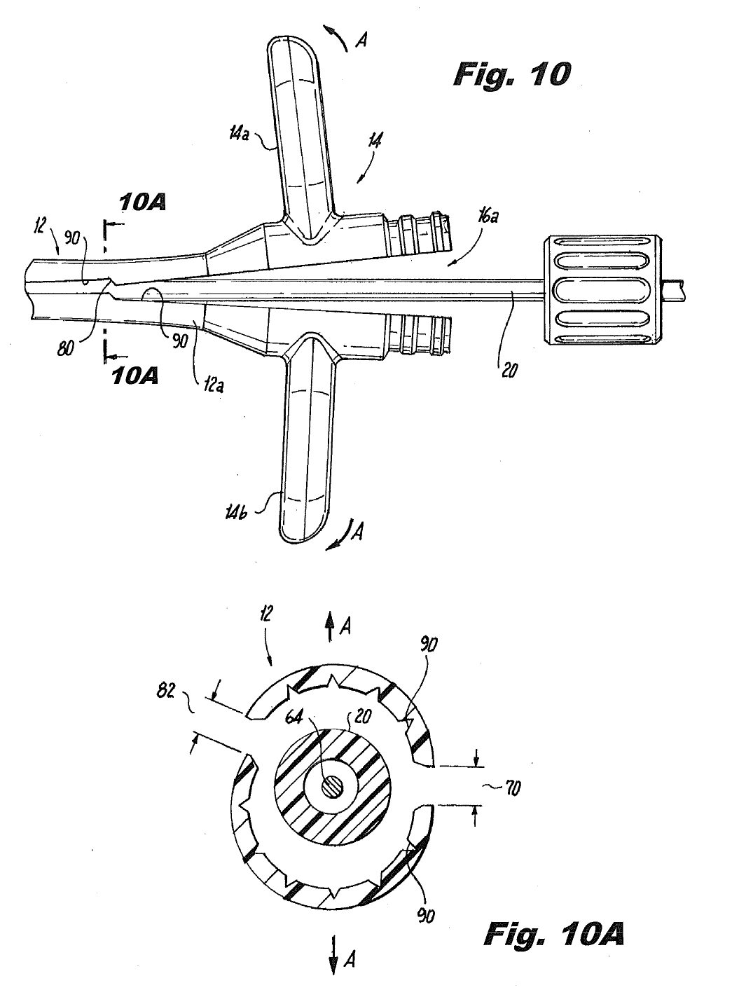

As shown in FIG. 9, in one exemplary embodiment, removal of the sheath 12 can be accomplished by pulling the splittable handles 14a, 14b outward from the longitudinal axis 38. When the handles 14a, 14b are pulled in the direction of arrow A, the proximal end of the sheath 12a will begin to split along a pair of peel lines perpendicular to line 9a-9a, as shown in FIG. 9A. FIG. 9A is a cross-sectional view of the sheath 12 illustrating the sheath being separated by a pair of peel lines perpendicular to line 9a-9a. The split as shown in FIGS. 9 and 9A show a split 70 on the peel lines 90 that are approximately in line with the central parting line 16a of the handles 14a, 14b. However, if the user peels the sheath 12 in a non-conventional way, such as holding one handle 14a and peeling the other handle 14b off center, the actual peel may not follow the peel line 90 and will instead tear to one side as shown in FIG. 10 described below. In one exemplary embodiment, a pair of peel lines 90 do not substantially line up with the central parting line 16a.

FIG. 10 is a perspective view of sheath 12 of FIG. 1, illustrating the sheath 12 being separated along a pair of peel lines 90 with a tear line crossing over to an alternate peel line 90. As shown, a tear 80 is formed during the further separation of the handles 14a, 14b.

In conventional sheaths, such a tear would prevent the sheath from peeling completely and the remainder of the sheath would have to be removed by cutting. By contrast, as shown in FIG. 10, if a tear 80 develops in sheath 12, the tear runs to an adjoining peel line. As a result, the sheath 12 is always peeled completely. FIG. 10A is a cross-sectional view of the sheath 12 taken along line 10a-10a of FIG. 10, illustrating the peel line separation after a tear line 80 between peel lines 90. However, it is shown that the tear 80 is now along a first split 70 and a second split 82. It can be appreciated that depending on many variables, such as how the handles 14a, 14b are twisted or pulled apart, other tears can occur between different peel lines 90 and a successful removal of the sheath 12 is still accomplished without requiring the cutting away of the sheath 12.

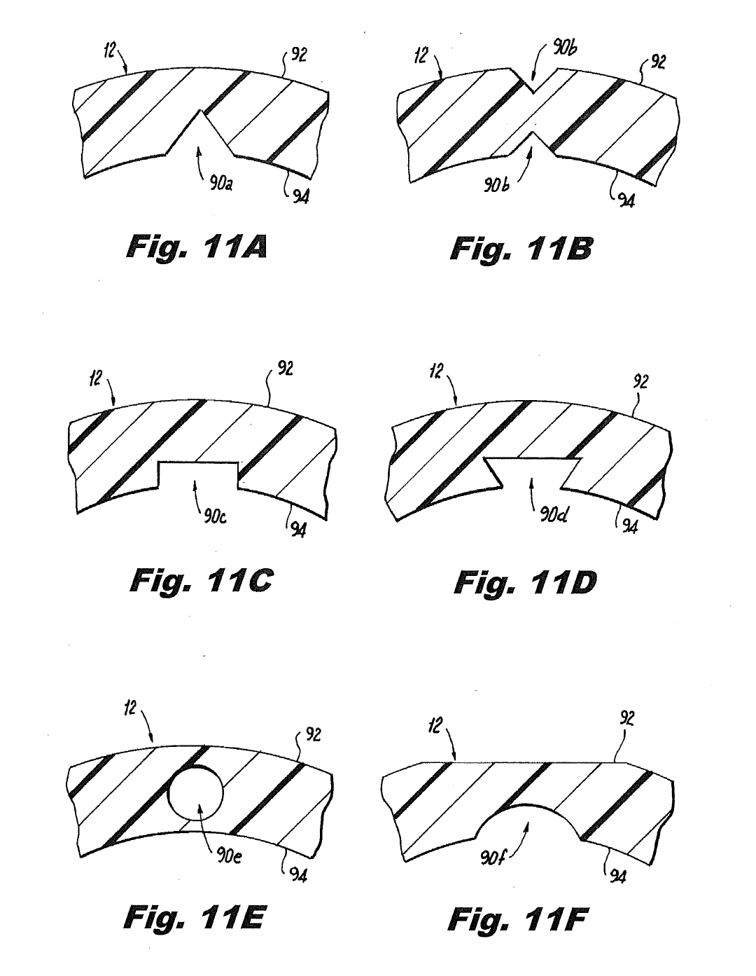

Referring now to FIGS. 11A-11F, cross-sectional views of alternative peelable introducer sheaths are shown. As will be appreciated by those of ordinary skill in the pertinent art, the sheaths utilize similar principles to the sheath 12 described above. Accordingly, the peel lines of the sheaths of FIGS. 11A-11F are appended with the letters a-f, respectively.

In FIG. 11A, the inner wall surface 94 of the sheath forms a v-shaped channel 90a with no modification of the outer wall surface. The channel 90a may come to a point such as an intersection between two lines or be a rounded shape such as a semi-circle. In one exemplary embodiment, the outer wall surface 92 can be substantially circular. The sheath of FIG. 11B has similar channel shaped inner wall surfaces 94 with corresponding channel shaped outer wall surfaces 92, both designated 90b. As a result, the peel line 90b can be made very thin. In one exemplary embodiment, the outer wall surface 92 peel line 90b can be a semicircular shape. In FIG. 11C, the inner wall surfaces of the sheath form a rectangular channel 90C with no modification of the outer wall surfaces.

In FIG. 11D, the inner wall surfaces of the sheath form a trapezoidal channel 90d also with no modification of the outer wall surface. In FIG. 11E, the peel lines of the sheath are formed by axial channels 90e within the wall of the sheath, between the inner and outer wall surfaces. As such, the sheath may appear unmodified except in cross-sectional view. In accordance with further aspects of the invention, the peel lines can be perforated lines.

As seen in FIG. 11F, each peel line 90f can be an axially extending region of reduced wall thickness defined by a concave inner wall surface and a corresponding concave or otherwise flattened outer wall surface. Alternatively, the peel lines may formed by perforations or any other frangible connection.

It is envisioned that the various wall formations can be mixed and matched as well as combined with other variations to form a desired peelable sheath.

In accordance with one aspect of the invention, the tubular sheath 12 of introducer assembly 10 is formed by an extrusion process using a polymeric material. Suitable materials from which the sheath 12 can be fabricated are, for example and not limitation, Polyether Block Amide (PEBA) manufactured by Arkema, Inc. under the trade name Pebax®, high-density polyethylene (HDPE), low-density polyethylene (LDPE), a blend of high-density polyethylene and low-density polyethylene, or similar materials. During the extrusion process, the polymeric material is forced through a die, for example, by a continuously rotating mixer such as a screw or flighted barrel. The rotating motion can impart rotational movement that creates a pitch of the regions of reduced wall thickness, with respect to a longitudinal axis of the sheath.

While the device and manufacturing methods of subject invention have been shown and described with reference to preferred embodiments, those skilled in the art will readily appreciate that changes may be made thereto without departing from the scope of the subject invention as defined by the appended claims.

The invention claimed is:

1. A Peelable Vascular Introducer Comprising:
an elongated tubular sheath having opposed proximal and distal ends and at least three circumferentially spaced peel lines extending from the proximal end to the distal end; and
spreadable handles associated with the proximal end of the sheath configured to split the sheath along the peel lines.

2. The peelable vascular introducer of claim 1, wherein the sheath has twelve peel lines.

3. The peelable vascular introducer of claim 2, wherein the sheath has a diameter of about four French to about twenty-two French.

4. The peelable vascular introducer of claim 1, wherein the sheath has between eight and twenty peel lines.

5. The peelable vascular introducer of claim 1, wherein the sheath has between ten and fourteen peel lines.

6. The peelable vascular introducer of claim 1, wherein the peel lines are defined by areas of reduced material thickness formed in the sheath.

7. The peelable vascular introducer of claim 1, wherein the peel lines are defined in the sheath by scored regions.

8. The peelable vascular introducer of claim 1, wherein the peel lines are defined in the sheath by perforated regions.

9. The peelable vascular introducer of claim 1, wherein the sheath is fabricated from a polyether block amide material.

10. A peelable vascular introducer comprising:
an elongated tubular sheath having opposed proximal and distal ends, and at least three circumferentially spaced peel lines extending from the proximal end portion to the distal end portion with an axially extending pitch; and
spreadable handles associated with the proximal end of the sheath for facilitating splitting of the sheath along the peel lines.

11. The peelable vascular introducer of claim 10, wherein the axially extending pitch of the peel lines is between about five degrees and forty-five degrees.

12. The peelable vascular introducer of claim 10, wherein the sheath has twelve peel lines.

13. The peelable vascular introducer of claim 12, wherein the sheath has a diameter of about four French to about twenty-two French.

14. The peelable vascular introducer of claim 10, wherein the sheath has between eight and twenty peel lines.

15. The peelable vascular introducer of claim 10, wherein the sheath has between ten and fourteen peel lines.

16. The peelable vascular introducer of claim 10, wherein the peel lines are defined by areas of reduced material thickness formed in the sheath.

17. The peelable vascular introducer of claim 10, wherein the peel lines are defined in the sheath by scored regions.

18. The peelable vascular introducer of claim 10, wherein the peel lines are defined in the sheath by perforated regions.

19. The peelable vascular introducer of claim 10, wherein the sheath is fabricated from a polyether block amide material.
